(19) United States
(12) Patent Application Publication
(10) Pub. No.: US 2007/0073306 A1
(43) Pub. Date: Mar. 29, 2007

(54) CUTTING BLOCK FOR SURGICAL NAVIGATION

(76) Inventors: Ryan Lakin, Newton, NJ (US); Garrett Sheffer, Hoboken, NJ (US); Jim McKale, Leesburg, IN (US); Ryan Schoenefeld, Ft. Wayne, IN (US)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/532,254

(22) Filed: Sep. 15, 2006

Related U.S. Application Data
(63) Continuation-in-part of application No. 10/795,891, filed on Mar. 8, 2004.

(51) Int. Cl. A61F 5/00

(52) U.S. Cl. 606/87

(57) ABSTRACT

A bone cutting apparatus configured to guide a surgical instrument during a bone cutting procedure is provided. The apparatus is attachable to a bone and comprises a cutting guide having a resection slot and a fine adjustment block coupled thereto. The fine adjustment block has a first adjustment dial configured to change the position of the resection slot relative to a resection plane. The system may further include a tracking element that is detectable and trackable by a surgical navigation system.

### RELATED APPLICATIONS

This application is a continuation-in-part of U.S. patent application Ser. No. 10/795,891, filed Mar. 8, 2004, the disclosure of which is hereby incorporated by reference in its entirety.

### FIELD OF THE INVENTION

The present method, apparatus, and system generally relate to surgical removal of portions of bone, and more particularly to facilitating accurate location of a cutting path for guiding a cutting instrument when creating a cut through a portion of a bone.

### BACKGROUND

As a result of many different physiological conditions, it is often necessary to replace joints in various parts of the body with prosthetic components. Surgical procedures for preparing bones to receive such components typically require precise shaping of portions of the bones. For example, during knee joint replacement surgery, precise resection of the ends of the femur and tibia is necessary to achieve a very close match between the surfaces remaining after removal of portions of the bones, and the mating surfaces of the prostheses. Without such precision mating, the reconstructed knee may result in misalignment (alignment that differs from optimal alignment corresponding to the patient's physical characteristics) of the femur and the tibia. Such misalignment may have a variety of undesirable consequences including discomfort to the patient, reduced mobility, and excessive wear on surfaces of the prostheses.

Cutting guides are commonly used to aid the surgeon by providing a surface across which a cutting instrument is moved to create a planar cut through a bone. Such guides permit the surgeon to achieve increased accuracy as compared to free hand bone shaping. The accuracy of the planar cut, however, is dependent upon accurate placement of the cutting guide. Therefore, it is desirable to provide a system for achieving highly accurate placement of a cutting guide to ensure precision mating between the planar cuts made through a bone and the corresponding surfaces of the prosthetic component. Accordingly, it would be desirable to overcome these and other shortcomings of the prior art.

### SUMMARY OF THE INVENTION

The present method, apparatus, and system (hereinafter collectively referred to as “the present system”) provides, in one embodiment, a drill cylinder having a body that defines a central bore, and an element configured to be detected by an image guidance system to permit image guidance of the drill cylinder to predetermined, target locations on the bone. Using the image guided drill cylinder, the surgeon may monitor on a display the current position and alignment of the drill cylinder body as compared to the target locations, and create bores into the bone at the target locations by inserting a drill bit through the central bore of the body. Alternatively, the surgeon may insert pins through the central bore into the bone at the target locations.

The present system further provides a cutting block having a frame, a guide adjustably connected to the frame, an adjustor connected to the frame, and mounting locations defined by the frame and configured to attach to the bone at the target locations. The mounting locations include either bores for receiving the pins that were inserted into the target locations, or pins for insertion into the bores that were created using the image guided drill cylinder. Accordingly, the cutting block can be accurately placed on the bone at the target locations, which in turn accurately places the guide.

The guide defines a cutting path through which a cutting instrument is passed to create a planar cut in the bone. The position of the guide (and the cutting path) relative to the mounting locations is adjustable using the adjustor. Depending upon the embodiment, actuation of the adjuster causes linear or angular adjustment of the position and/or orientation of the cutting path relative to the mounting locations.

Additionally, the present system may include a tracking instrument having an element configured to be detected by the image guidance system and an engagement portion configured to engage the cutting path to permit image guided adjustment of the cutting path. Thus, in addition to providing accurate placement of the cutting path using the image guide drill cylinder to locate the mounting locations of the cutting block, the present system enables the surgeon to achieve even greater accuracy by providing image guided adjustment of the cutting path when the cutting block is mounted to the bone.

In one form thereof, there is provided a bone cutting apparatus configured to guide a surgical instrument during a bone cutting procedure. The apparatus is attachable to a bone and comprises a cutting guide having a resection slot and a fine adjustment block coupled thereto. The fine adjustment block has a first adjustment dial configured to change the position of the resection slot relative to a resection plane. The system may further include a tracking element that is detectable and trackable by a surgical navigation system.

In another form thereof, a method of fine adjusting a bone cutting apparatus during a surgical navigation procedure is provided. The method comprises attaching a cutting guide assembly to a bone, wherein the cutting guide assembly has a resection slot. The position of the resection slot is adjusted relative to a predetermined resection plane by moving at least one adjustment dial on the cutting guide assembly. The resection slot is then aligned with the predetermined resection plane by image guiding a tracking element associated with the cutting guide assembly.

In yet another form thereof, a method of fine adjusting an instrument during a surgical navigation procedure is provided. The method comprises positioning a drill guide assembly against a bone, wherein the drill guide assembly has at least one drill hole. A surgical tool having a tracking element detectable by a surgical navigation system is incorporated into the drill guide assembly, and the position of the drill guide assembly is adjusted so that the at least one drill hole substantially corresponds with a target location on the bone. One or more holes are drilled into the bone at the target location, and one or more guide pins are inserted into the one or more drilled holes. A resection instrument is then placed over the one or more guide pins.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a perspective view of one embodiment of a cutting block for use with the present system;

FIG. 2 is a top, plan view of the cutting block of FIG. 1;

FIG. 3 is a partially fragmented, side elevation view of a portion of the cutting block of FIG. 1;

FIG. 4 is a partially fragmented, perspective view of another portion of the cutting block of FIG. 1;

FIG. 5 is a perspective view of one embodiment of a drill cylinder for use with the present system;

FIG. 6 is a perspective view of embodiments of tracking instruments for use with the present system;

FIG. 7 is a combination of a perspective view of the drill cylinder of FIG. 5 being image guided toward a target location on a bone, and a conceptual diagram of an image guidance system for use with the present system;

FIG. 8 is a side elevation view of the cutting block of FIG. 1 mounted to a bone in a position to make a distal femoral cut;

FIG. 9 is a perspective view of the drill cylinder of FIG. 5 being image guided toward a target location on a bone;

FIG. 10 is a side elevation view of the cutting block of FIG. 1 mounted the distal end of a femur and having a tracking instrument of FIG. 6 mounted thereto;

FIG. 11 is a perspective view of the drill cylinder of FIG. 5 being image guided toward a target location on a bone;

FIG. 12 is a side elevation view of the cutting block of FIG. 1 mounted to a bone in a position to make a proximal tibial cut;

FIG. 13 is a perspective view of the drill cylinder of FIG. 5 being image guided toward a target location on a bone;

FIG. 14a is a perspective view of a portion of an exemplary fine adjustment bone cutting apparatus in accordance with the present teachings;

FIG. 14b is a perspective view of an exemplary fine adjustment bone cutting apparatus in accordance with the present teachings;

FIG. 14c is a side view of the exemplary fine adjustment bone cutting apparatus of FIG. 14b;

FIG. 14d is a cross-sectional view of a portion of the fine adjustment bone cutting apparatus of FIG. 14b;

FIG. 15a is a perspective view of another exemplary fine adjustment bone cutting apparatus in accordance with the present teachings;

FIGS. 15b-c depict axial movement embodiments of the fine adjustment bone cutting apparatus of FIG. 15a;

FIGS. 15d-e depict rotational movement embodiments of the fine adjustment bone cutting apparatus of FIG. 15a;

FIG. 15f depicts a ratchet locking mechanism in accordance with the present teachings;

FIG. 16a is a perspective view of a fine adjustment bone cutting apparatus being surgically navigated in accordance with the present teachings;

FIG. 16b is a perspective view of guide pins being inserted into the bone cutting apparatus of FIG. 16a;

FIG. 16c is a perspective view of an exemplary bone cutting guide fitted over the guide pins of FIG. 16b;

FIG. 17a is a perspective view of another fine adjustment bone cutting apparatus in accordance with the present teachings;

FIG. 17b depicts the fine adjustment bone cutting apparatus of FIG. 17a being fine adjusted by surgical navigation techniques;

FIGS. 17c-d depict an exemplary outrigger device being incorporated into the fine adjustment bone cutting apparatus of FIG. 17a;

FIG. 18 depicts an exemplary cut block manipulation device in accordance with the present teachings;

FIG. 19a is a fragmentary perspective view of a drill positioned to form a hole in a bone by using a drill guide tracked by surgical navigation in accordance with the present teachings; and

FIG. 19b depicts an exemplary cutting block assembly being fine adjusted by a surgical navigation process in accordance with the present teachings.

Corresponding reference characters indicate corresponding parts throughout the several views.

### DETAILED DESCRIPTION

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

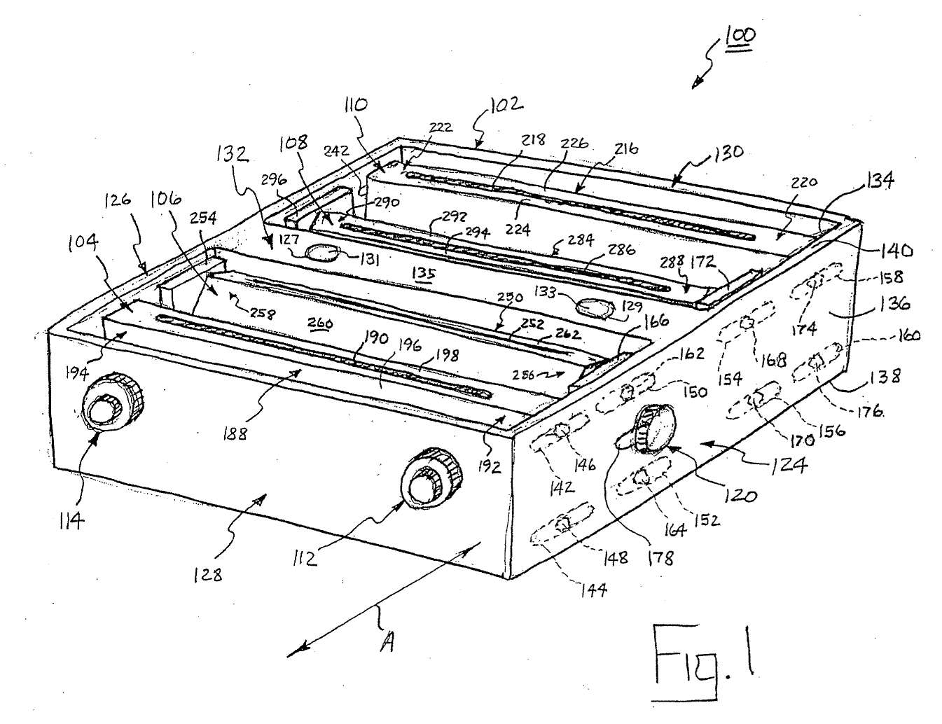

Referring now to FIGS. 1-3, one embodiment of a cutting block 100 for use in the present system is shown. Cutting block 100 generally includes a frame 102, four adjustable guides 104, 106, 108, 110, four linear adjustors 112, 114, 116, 118, and two angular adjustors 120, 122. It should be understood that the number of guides and adjustors may be different from the number described herein. More specifically, one skilled in the art could readily adapt the teachings of this disclosure to provide a cutting block with one, two, three, or more than four guides with a corresponding number of linear and angular adjustors to position the guides as taught herein. For example, a single guide may be provided that is adjustable within frame 102 into four different positions for making each of the bony cuts described below. In this instance, the single guide would be adjustable linearly and angularly as will become apparent from the following description of embodiments of the system. Alternatively, a pair of guides may be used, such as guides 104, 110, each being adjustable linearly and angularly. Of course, three linearly adjustable guides may be used in a different embodiment, wherein one or more of the guides is also angularly adjustable.

Additionally, it should be understood that while the following description discusses, for example, linear adjustors configured to adjust the position of one end of two guides, separate linear adjustors may be used for each guide. Each of the separate linear adjustors may be configured to adjust both ends of the guide corresponding to the adjustor. Moreover, the described combined adjustor may readily be adapted to adjust both ends of both guides, or both ends of one guide and one end of the other guide. Finally, the described angular adjustors may readily be configured by one skilled in the art to simultaneously adjust the angle of orientation of two or more guides. These and other variations of the guide/adjustor configuration are contemplated by this disclosure and encompassed by the appended claims.

In one embodiment of the present system, frame 102 of cutting block 100 includes a pair of parallel side walls 124, 126 and a pair of parallel end walls 128, 130 that extend substantially perpendicular to side walls 124, 126 to form the substantially rectangular perimeter of frame 102 shown in the Figures. While a rectangular configuration is shown in the Figures, frame 102 may be provided in any configuration suitable for supporting guides 104-110 and accommodating their adjustment. An attachment wall 132 also extends perpendicularly between side walls 124, 126 at a location between end walls 128, 130. Attachment wall 132 may alternatively be replaced by a pair of extensions (one from side wall 124 and one from side wall 126), or omitted in an embodiment wherein side walls 124, 126 or end walls 128, 130 are adapted to facilitate attachment of frame 102 to the target bone. In the illustrated embodiment, attachment wall 132 includes two mounting locations 127, 129, which in the embodiment shown, are occupied by two bores 131, 133, respectively, that extend from an upper surface 135 of attachment wall 132 to a lower surface 137 of attachment wall 132. As is further described below, bores 131, 133 may function as drill guides during attachment of cutting block 100 to a bone, and receive pins after bores are formed in the bone to secure cutting block 100 to the bone. Alternatively, the pins may be placed percutaneously as part of a minimally invasion surgery (MIS) procedure, and secured to the bone to receive bores 131, 133 of cutting block 100. In yet another alternative embodiment, as is also described below, mounting locations 127, 129 may instead be occupied by a pair of pins that extend from lower surface 137 and into bores formed in a bone, thereby securing cutting block 100 to the bone. So long as an adequate connection is provided between cutting block 100 and the target bone, one or more than two of either type of mounting location 127, 129 configuration may be used.

As best shown in FIG. 1, each side wall 124, 126 includes a plurality of travel guides or channels configured to accommodate movement of guides 104-110 during adjustment as further described herein. Since side wall 126 is substantially a mirror image of side wall 124, only side wall 124 is described in detail. Side wall 124 includes an interior surface 134, an exterior surface 136, a lower surface 138, and an upper surface 140. A first upper channel 142 is formed into interior surface 134 and is elongated in direction A as shown in FIG. 1. While each of the plurality of channels is described as being formed into side wall 124, it should be understood that the channels may readily be replaced by ridges that extend inwardly from interior surface 134 to form a pair of guide rails. Alternatively, as will become apparent from the following description, the channels may be replaced with posts or protrusions of some other configuration that extend from interior surface 134 and move within channels or guide rails formed on the corresponding guides 104-110. In yet another alternative embodiment, a single channel or guide rail is provided (on either interior surface 134 or guide 104), and configured to accommodate movement of a single post (on either interior surface 134 or guide 104).

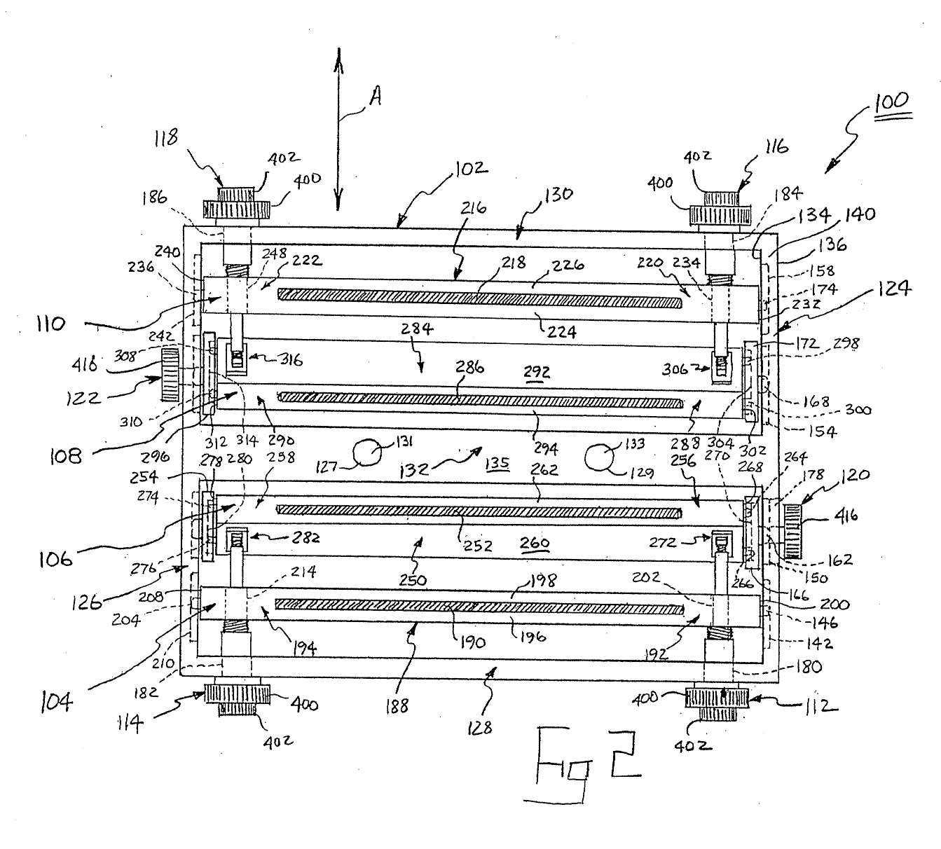

Referring to both FIGS. 1 and 2, in the depicted embodiment, a first lower channel 144 (FIG. 1) is also formed into interior surface 134 of side wall 124, and extends in direction A in substantially parallel relationship to upper channel 142. First upper channel 142 and first lower channel 144 are configured to receive an upper post 146 and a lower post 148, respectively, of adjustable guide 104 as is further described below. Side wall 124 further includes a second upper channel 150, a second lower channel 152, a third upper channel 154, a third lower channel 156, a fourth upper channel 158, and a fourth lower channel 160. Second channels 150, 152 are configured to receive a pair of posts 162, 164, respectively, extending from a mounting plate 166 associated with adjustable guide 106. Similarly, third channels 154, 156 are configured to receive posts 168, 170 of mounting plate 172 associated with adjustable guide 108. Likewise, fourth channels 158, 160 are configured to receive posts 174, 176 of adjustable guide 110. Finally, side wall 124 also includes a slot 178 positioned in substantially parallel relationship between second channels 150, 152. As will become apparent from the description below, slot 178 accommodates movement of angular adjustor 120 when adjustable guide 106 is adjusted in direction A.

End wall 128 includes an opening 180 configured to receive a portion of linear adjustor 112, and an opening 182 configured to receive a portion of linear adjustor 114. Similarly, end wall 130 includes openings 184, 186 configured to receive portions of adjustors 116, 118, respectively.

Guide 104 generally includes a body 188 having at least one surface defining a cutting path 190. In the embodiment shown, cutting path 190 is defined by facing surfaces of a pair of substantially parallel guide walls 196, 198 and is sized to receive the blade of a surgical saw or other cutting instrument (not shown) as is further described below. It should be understood, however, that a single surface of a single guide wall may be used to define any of the cutting paths described herein. Body 188 has a substantially rectangular cross-section and extends substantially the entire distance between interior surfaces 134 of side walls 124, 126. Body 188 includes a first end portion 192, and a second end portion 194, between which extend guide walls 196, 198 to define path 190. First end portion 192 includes spaced apart posts 146, 148 (described above), which extend from an end surface 200 of end portion 192. Additionally, in the embodiment shown, a bore 202 extends through end portion 192 to permit a portion of linear adjustor 112 to extend to guide 106 as is further described below. Second end portion 194 similarly includes a pair of spaced apart posts 204, 206 (only post 204 is shown), which extend from an end surface 208 of end portion 194. Posts 204, 206 also extend into a pair of appropriately spaced channels 210, 212 (only channel 210 is shown) formed in side wall 126 (similar to channels 142, 144 of side wall 124). Finally, end portion 194 also includes a bore 214 (like bore 202 of end portion 192) to permit a portion of adjustor 114 to extend to guide 106 as is further described below.

Guide 110 is substantially identical to guide 104, including all of the same components, in substantially the same configuration. These components include a body 216, a cutting path 218, a first end portion 220, a second end portion 222, and a pair of parallel guide walls 224, 226 having facing surfaces that define cutting path 218. First end portion 220 includes spaced apart posts 174, 176 (described above) extending from an end surface 232, and a bore 234 to receive a portion of adjustor 116. Second end portion 222 also includes a pair of posts 236, 238 (only post 236 is shown) extending from an end surface 240 into a pair of appropriately spaced channels 242, 244 (only channel 242 is shown) formed in side wall 126, and a bore 248 to receive a portion of adjustor 118. Both of guides 104, 110 (and corresponding cutting paths 190, 218) are supported between side walls 124, 126 in a substantially vertical orientation relative to lower surfaces 138 of side walls 124, 126 to guide a surgical saw (not shown) in making bony cuts that are substantially perpendicular to direction A as is further described below.

As best shown in FIG. 2, guide 106 generally includes a body 250 having at least one surface defining a cutting path 252 that receives the blade of a surgical saw (not shown) to create a chamfer cut as is further described below. In the embodiment shown, body 250, like body 188 and body 216, has a substantially rectangular cross-section. Body 250 extends between mounting plate 166 and a similar mounting plate 254 positioned adjacent side wall 126. Body 250 includes a first end portion 256, a second end portion 258, and a pair of parallel guide walls 260, 262 that extend between end portions 256, 258 to define cutting path 252. First end portion 256 includes a pair of spaced apart posts 264, 266, which extend from an end surface 268 of end portion 256 into an arcuate channel 270 formed in mounting plate 166 as is further described below. It should be understood, however, that a pair of arcuate channels (one that receives post 264, and one that receives post 266) may be formed in mounting plate 166. Alternatively, posts 264, 266 may be mounted to mounting plate 166 for cooperation with a corresponding channel (or channels) formed on end surface 268. Moreover, it should be understood that a single post/channel configuration may readily be employed. Any of these alternatives are suitable for use at either end of either of guides 106, 108 so long as they facilitate angular adjustment of cutting paths 252, 286 as described below.

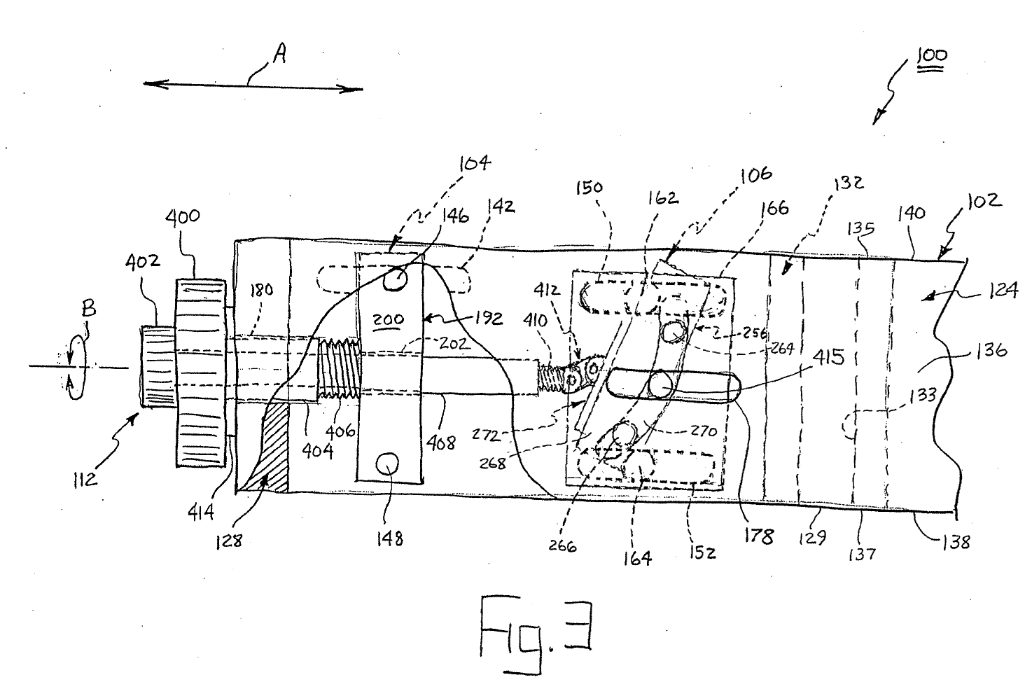

Still referring to the embodiment depicted in FIG. 2, end portion 256 further includes a drive bracket, generally designated by the number 272, which is connected through a linkage to adjustor 112 as is described in greater detail below with reference to FIG. 3. Similarly, second end portion 258 includes a pair of spaced apart posts 274, 276, which extend from an end surface 278 of end portion 258 into an arcuate channel 280 formed in mounting plate 254, and a drive bracket 282, which is connected through a linkage to adjustor 114.

Guide 108 is substantially identical to guide 106, including all of the same components, in substantially the same configuration. The components include a body 284, a cutting path 286, a first end portion 288, a second end portion 290, and a pair of parallel guide walls 292, 294. Body 284 extends between mounting plate 172 and a similar mounting plate 296 positioned adjacent side wall 126. First end portion 288 includes a pair of posts 298, 300, which extend from an end surface 302 into an arcuate channel 304 formed in mounting plate 172, and a drive bracket 306 linked to adjustor 116. Second end portion 290 includes a pair of posts 308, 310, which extend from an end surface 312 into a similar arcuate channel 314 formed in mounting plate 296, and a drive bracket 316 linked to adjustor 118.

Each of linear adjustors 112-118 is substantially identical. Accordingly, only adjustor 112 is described in detail herein with reference primarily to FIG. 3. It should be understood that a variety of different adjustment mechanisms may be employed consistent with the teachings of this disclosure to facilitate adjustment of guides 104-119 in direction A. Adjustor 112 is described as a manually operated, mechanical adjustment mechanism. Alternatively, an electrical adjustment mechanism may be used having a motor or other force-providing device with an actuator (such as a button, switch, etc.) either mounted to frame 102 or remotely located therefrom. It should also be understood that the threaded post and cylinder arrangement described below is merely illustrative, and may readily be replaced with any one of a plurality of different known types of linear adjustment mechanism. Additionally, while adjustment mechanisms that convert rotational motion into linear motion are described below, any other type of suitable mechanism may be used so long as it facilitates adjustment of at least a portion of guides 104-110 (and cutting paths 190, 218, 252, 286) in direction A.

Referring now to FIG. 3, adjustor 112 generally includes a pair of grips 400, 402, a first internally threaded shaft 404, an externally threaded cylinder 406 attached to first end portion 192 of guide 104, a second internally threaded shaft 408, a threaded rod 410, and a linkage 412 attached between threaded rod 410 and drive bracket 272 of guide 106. In the illustrated embodiment, grips 400, 402 are in the form of knobs. It should be understood, however, that grips 400, 402 could readily be formed as plates, sliders, or any other suitable structure configured to cause movement of end portion 192 of guide 104 and end portion 256 of guide 106 in the manner described below. Grip 400 is shown as a large diameter knob, rigidly connected to first internally threaded shaft 404 for rotation therewith in direction B about a central axis of adjustor 112. Grip 400 may be spaced from end wall 128 by a spacer 414. Grip 402 is shown as a smaller diameter knob, rigidly connected to second externally threaded shaft 408, for rotation therewith in direction B. In the illustrated embodiment, grip 402 and shaft 408 rotate independent of grip 400 and shaft 404.

Shaft 404 extends through bore 180 of end wall 128. A conventional retaining mechanism may be used to prevent both grips 400, 402 from moving outwardly, away from end wall 128, while permitting rotation of shafts 404, 408. As should be apparent from FIG. 3, shaft 408 extends with sufficient clearance for rotation through an interior bore (not shown) of shaft 404, an interior bore (not shown) of threaded cylinder 406, and bore 202 through guide 104. Cylinder 406 includes external threads that mate with the internal threads (not shown) of shaft 404. As a result, when grip 400 and shaft 404 are rotated in direction B, cylinder 406 (and guide 104 connected thereto) is moved toward or away from mounting locations 127, 129, depending upon the direction of rotation. As cylinder 406 and guide 104 move, posts 146, 148 move within channels 142, 144, respectively, which support guide 104 in the substantially vertical orientation shown in FIG. 3.

Threaded rod 410 similarly includes external threads that mate with the internal threads (not shown) of shaft 408. Threaded rod 410 is connected to bracket 272 of end portion 256 through linkage 412. Linkage 412 is shown as a pair of pivotal connections which permit tilting of guide 106 (as is further described below). Of course, one of ordinary skill in the art could readily implement any suitable linkage or joint configuration that simultaneously facilitates movement of guide 106 in direction A and tilting of guide 106. As grip 402 and shaft 408 are rotated in direction B, threaded rod 410 is threaded into or out of shaft 408, thereby moving rod 410, linkage 412, and guide 106 toward or away from mounting locations 127, 129, depending upon the direction of rotation. As guide 106 moves, mounting plate 166 also moves, since posts 264, 266 are captured by channel 270 formed in mounting plate 166. Consequently, posts 162, 164 of mounting plate 166 travel within channels 150, 152, respectively, which support mounting plate 166 in the vertical orientation shown in the Figure. Guide 106 is thus maintained in its selected tilted or angular orientation while being moved toward or away from mounting locations 127, 129. It should further be understood that the rod 415 connected to angular adjustor 120 also moves with guide 106. During its travel, rod 415 is guided by slot 178 formed through side wall 124 of frame 102.

Referring now to FIG. 4, one embodiment of angular adjustor 120 will be described. Since angular adjustors 120, 122 are substantially identical, only angular adjustor 120 is described in detail below. Angular adjustor 120 includes a grip 416 connected to one end of rod 415, a gear 418 connected to the other end of rod 415, and a track 420 connected to end surface 268 of guide 106. As shown, rod 415 extends from grip 416, through slot 178 of side wall 124, through an opening 422 formed through mounting plate 166, and is rigidly connected to gear 418. Gear 418 includes teeth 424. Track 420 is formed such that the teeth 426 of track 420 curve in a manner corresponding to the curve of arcuate channel 270 of mounting plate 166. As should be apparent from the Figure, teeth 424 of gear 418 are positioned in meshing engagement with teeth 426 of track 420.

In operation, grip 416 of angular adjustor 120 is rotated in direction C about a central axis of adjustor 120. As grip 416 rotates, rod 415 rotates, thereby causing rotation of gear 418. As gear 418 rotates, the engagement of teeth 424 and teeth 426 causes guide 106 to move upwardly or downwardly, depending upon the direction of rotation. A portion of the upward or downward movement is translated into a tilt or angular adjustment of the orientation of guide 106 (and cutting path 190) because posts 264, 266 are captured within arcuate channel 270. It should be understood that any suitable locking mechanism may be employed to retain adjustor 120 in position after the orientation of guide 106 has been adjusted. As explained above, the position of guide 106 may also be linearly adjusted using, for example, linear adjustor 112. When linear adjustor 112, which is connected to guide 106 through drive bracket 272 (not shown in FIG. 4), is operated, guide 106, mounting plate 166, and angular adjustor 120 are moved in direction A toward or away from mounting locations 127, 129. During movement in direction A, posts 162, 164 (only post 164 is shown in FIG. 4) move within channels 150, 152 (only channel 152 is shown in FIG. 4), and rod 415 moves within slot 178.

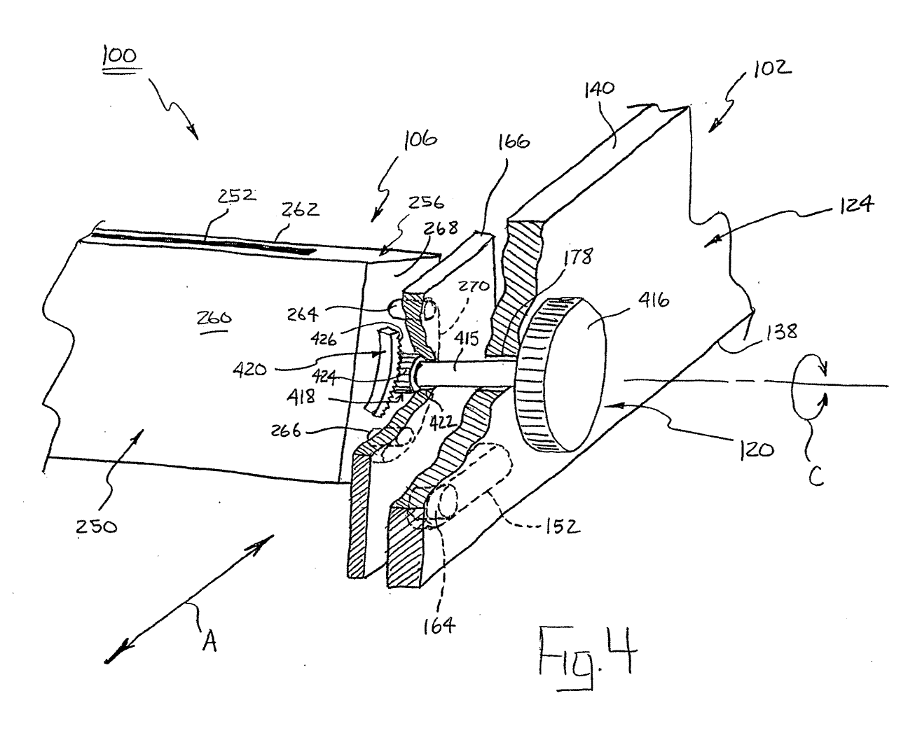

Referring now to FIG. 5, one embodiment of an image-guided drill cylinder 500 is shown. Drill cylinder 500 generally includes a body 502, a handle 504, and an image-guidance assembly 506. Body 502 includes a first end 508, a second end 510, and a cylindrical outer wall 512 extending between first and second ends 508, 510 to define a central bore 514. Although outer wall 512 is shown as a solid wall having a substantially constant cross-section, outer wall 512 may instead include openings or open areas and have more than one diameter dimension.

Handle 504 includes a rod 516 and a grip 518. Rod 516 is connected at one end to body 502 of drill cylinder 500, and at the other end to grip 518. It should be understood that the connection between body 502 and rod 516 may be permanent or provide for disconnection of rod 516 (and therefore handle 504). In such an embodiment, handle 504 may be attached to any of a plurality of drill cylinders 500, each having a central bore 514 sized to receive a different size drill bit or fastener, as is further explained below.

Image-guidance assembly 506 includes a shaft 520 connected at one end to body 502 of drill cylinder 500, and at the other end to an array 522 including a plurality of detectable elements 524, 526, 528 connected together by a frame 530. While three detectable elements 524-528 are shown, it should be understood that one, two, or more than three such elements may readily be employed by one skilled in the art, consistent with the teachings of this disclosure. Each detectable element 524-528 is configured to either emit a location signal (e.g., an RF signal, an IR signal, etc.) that is detectable by a receiver as is further described below. Elements 524-528 may utilize passive technology wherein the location signals are only emitted when elements 524-528 are excited by an external source (not shown), or an active technology wherein elements 524-528 emit the location signals so long as a power source (e.g., a battery (not shown)) provides power to elements 524-528. Frame 530 maintains elements 524-528 in fixed relationship to one another. Additionally, the position of array 522 relative to first end 508 of body 502 is also fixed. Thus, by sensing the position of array 522, a conventional image guidance system 808 (described below) may be programmed to determine the precise location of first end 508 of body 502 relative to a fixed reference point as is further described below.

In an alternate embodiment, image-guidance assembly 506 is mounted directly to a drill (not shown) instead of to body 502. In this embodiment, shaft 520 is connected at one end to the drill, and at the other end to array 522. Image-guidance assembly 506 is attached to drill in a known location, such that detection of the position of array 522 enables a determination (by an image guidance system 808 as described below) of the position, relative to a fixed reference point, of an end of the drill bit of the drill, and of the orientation of an axis of the drill bit.

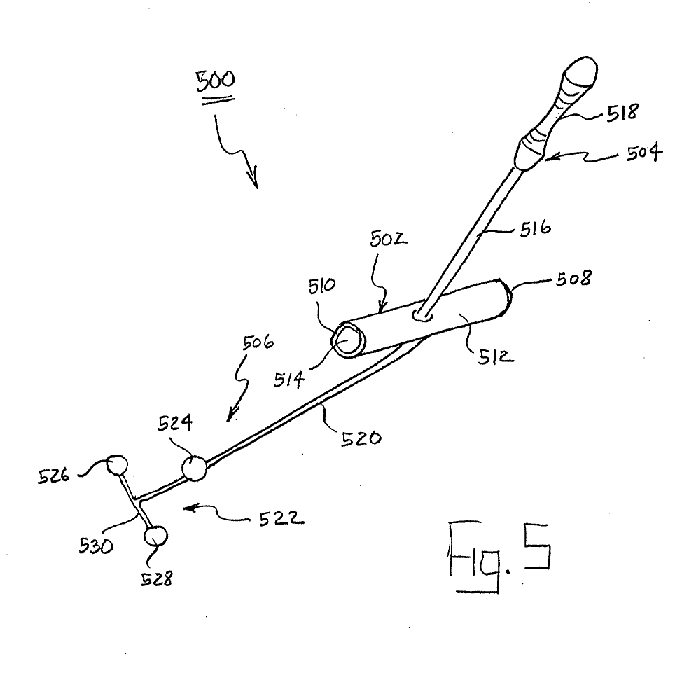

FIG. 6 illustrates two embodiments of tracking instruments for use with the present system. Tracking instrument 600 generally includes a grip 602, an array 604 connected to grip 602 by a shaft 606, and a probe 608. Array 604 is essentially identical to array 522 of drill cylinder 500, including a plurality of detectable elements 610, 612, 614 connected together by a frame 616. Thus, the comments provided above regarding array 522 apply equally to array 604. Probe 608 includes a shaft 618 connected at one end to grip 602, and having at the opposite end an engagement portion or tip 620. As should be apparent from the foregoing, by detecting the position of array 604, image guidance system 808 may be programmed to determine the precise location of tip 620 relative to a reference point since the relative locations of array 604 and tip 620 are fixed.

The tracking instrument 700 shown as the second embodiment in FIG. 6 is substantially identical to tracking instrument 600, except that tip 620 is replaced with an engagement portion formed in the shape of a plate 720 attached to the end of shaft 718. Accordingly, the remaining components of tracking instrument 700 are not shown. As is described in greater detail below, plate 720 is sized to fit within or on cutting paths 190, 252, 286, 218 of guides 104-110, respectively. In this manner, since the relative locations of array 704 (not shown) and plate 720 are known, the precise location of the cutting paths 190, 252, 286, 218 (relative to a reference point) may be determined by image guidance system 808.

Having described the various components of embodiments of the present system, the following portion of the specification discusses examples of applications of the system. For example, the system may be used in a total knee replacement or arthroplasty procedure as described below. It should be understood, however, that the teachings provided herein are also applicable to various other surgical procedures involving removal of portions of bone to, for example, prepare a joint to receive a prosthetic implant.

If applied in an arthroplasty procedure, the present system facilitates removal of portions of femoral and tibial bone to prepare the bone surfaces for the femoral and tibial prosthetic implants, respectively. First, the distal end of the femur and the proximal end of the tibia are surgically exposed in a conventional manner. The knee joint is then flexed to fully expose the distal end 800 of the femur 802. Referring to FIG. 7, image-guided drill cylinder 500 is next moved by the surgeon into a desired location 804 for placing a headless pin, post, or other similar device (hereinafter referred to as a pin), which in turn locates cutting block 100. While FIG. 7 depicts an entirely exposed bone structure, it should be understood that the pin placement described below may instead be accomplished percutaneously as part of an MIS procedure wherein a small incision is made at the desired pin location, image-guided drill cylinder 500 is placed through the small incision, and a bore is formed for placement of the pin in the manner described below. More specifically, while viewing a visual indication of the three-dimensional orientation of drill cylinder 500 relative to the structure of distal end 800 of femur 802 on a display 806 of an image guidance system 808, the surgeon moves drill cylinder 500 into contact with target location 804 on femur 802 (either directly or through a small incision in the skin), which corresponds to the precise, desired location of one of mounting locations 127, 129. In this example, target location 804 corresponds to the precise, desired location of one of bores 131, 133 of attachment wall 132 of cutting block 100. Image guidance system 808 provides a visual indication on display 806 based on data representing the three-dimensional structure of femur 802, drill cylinder 500, and cutting block 100. A receiver 810 of image guidance system 808 detects the position of elements 524-528 of array 522 connected to body 502 of drill cylinder 500, thereby enabling system 808 to accurately determine the location of first end 508 of body 502 relative to femur 802. In this manner, the surgeon is able to determine not only whether the point of entry of the drill bit (not shown) is accurately located, but whether the angle of entry (represented by axis D of FIG. 7) is appropriate for placement of the pin to be received by cutting block 100.

Once drill cylinder 500 is accurately placed, an appropriately sized pin is placed into central bore 514 of drill cylinder 500 and screwed or otherwise inserted into target location 804 of femur 802. The depth of entry of the pin may be controlled using any of a variety of conventional techniques. After the pin is placed, drill cylinder 500 is removed. It should be understood, however, that instead of placing a pin into femur 802 as described above, drill cylinder 500 may be used to guide a drill bit (not shown) for creating a bore into femur 802. Alternatively, using the embodiment described above wherein array 506 is connected to a drill, the drill may be used directly, under image guidance, to create a bore in target location 804 of femur 802. In either case, the depth of entry of the drill bit may similarly be controlled using any of a variety of conventional techniques. After the bore is created and drill cylinder 500 is removed, an appropriately sized pin may be securely inserted into the bore. The corresponding cutting block 100 in such an embodiment includes bores 131, 133 through attachment wall 132 at mounting locations 127, 129, respectively. Bores 131, 133 are sized to securely receive such pins. As yet another alternative, drill cylinder 500 may be used in the manner described above to create a bore in femur 802 for receiving a pin connected to cutting block 100 at one of mounting locations 127, 129.

Assuming a first pin is placed into femur 802 according to the procedure described above, a second pin may be placed by repeating the procedure, but placing the second pin at a second target location (not shown) that corresponds to a second bore 131, 133 in the known geometry of cutting block 100. After the second pin is inserted into the second target location, drill cylinder 500 is removed. Next, cutting block 100 is positioned onto the first and second pins such that the pins are received by bores 131, 133 formed in attachment wall 132. Any of a plurality of different techniques may be used to secure cutting block 100 to the pins. For example, the pins may include threaded ends that extend from femur 802, through cutting block 100, and project beyond surface 135 of mounting wall 132. The projecting portions of the threaded ends may receive nuts or similar components which may be tightened onto the threaded ends against surface 135. In this manner, cutting block 100 is securely captured in a fixed orientation between the nuts and the femur. Alternatively, cutting block 100 may include set screws or similar components which are controllably inserted into the bores and urged (such as by rotation on internal threads of cutting block 100) into locked engagement with a surface of each pin. Of course, any other connection technique for securely attaching cutting block 100 to the pins is within the scope of the teachings provided herein.

In an alternative embodiment, after the first pin is placed according to the procedure described above, one bore, such as bore 131, of cutting block 100 is placed onto the first pin, but cutting block 100 is not tightly secured to the pin. The second pin may be placed by placing drill cylinder 500 over another bore, such as bore 133, in a predetermined placement location, and rotating cutting block 100 about the first pin until display 806 indicates that drill cylinder 500 is accurately positioned over the second target location on femur 802. After drill cylinder 500 is accurately located, the second pin is simultaneously inserted through drill cylinder 500 (in the manner described above) and bore 133 of cutting block 100. Alternatively, cutting block 100 placed on the first pin may include, or later have attached to it, a tracking instrument (such as instruments 600 or 700) to permit detection of the position of cutting block 100 (more specifically, the location of second bore 133 in cutting block 100). The surgeon may rotate cutting block 100 while viewing the location of second bore 133 on display 806. When second bore 133 is rotated into registration with the second target location on femur 802, the surgeon may secure cutting block 100 to the first pin to fix cutting block 100 in place. Then, a drill or other pin insertion device may be used to insert the second pin through second bore 133 of cutting block 100 and into the second target location of femur 802.

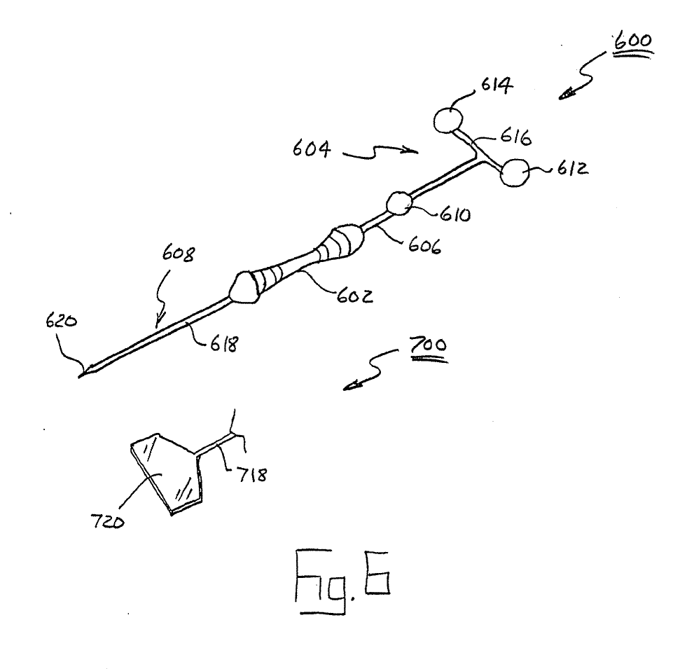

After cutting block 100 is secured to the distal end 800 of femur 802 as shown in FIG. 8, the alignment of the cutting path (such as cutting path 218 of guide 110) for creating a first cut (the distal femoral cut) may be adjusted to correspond with the appropriate anterior posterior plane (plane E). To this end, plate 720 of tracking instrument 700 may be inserted into path 218. The surgeon may then rotate grips 400 of adjustors 116, 118 in the manner described above to move end portions 220, 222 of guide 216, respectively, toward or away from mounting locations 127, 129, thereby positioning path 218 such that when a surgical saw is moved along path 218 it will create a surface corresponding to plane E at distal end 800 of femur 802 in the desired orientation, such as substantially perpendicular to the femoral mechanical axis. After plane E is created, cutting block 100 and pins are removed from femur 802.

Alternatively, instead of using tracking instrument 700 (or tracking instrument 600) to provide image guided adjustment of cutting path 218 as described above, plane E may simply be created using cutting block 100 without adjustments to the position of cutting path 218. After plane E is created, the surgeon may position plate 720 of tracking instrument 700 (or tip 620 of tracking instrument 600) onto plane E to verify the location and orientation of plane E by receiving feedback from the image guidance system described below. If plane E is not in a desired location and orientation, the surgeon may adjust the position of cutting path 218 as described above, make a new cut, and verify the accuracy of the cut by again placing plate 720 of tracking instrument 700 (or tip 620 of tracking instrument 600) onto the newly created plane. This iterative process may be repeated until the surgeon is satisfied with the accuracy of the cut.

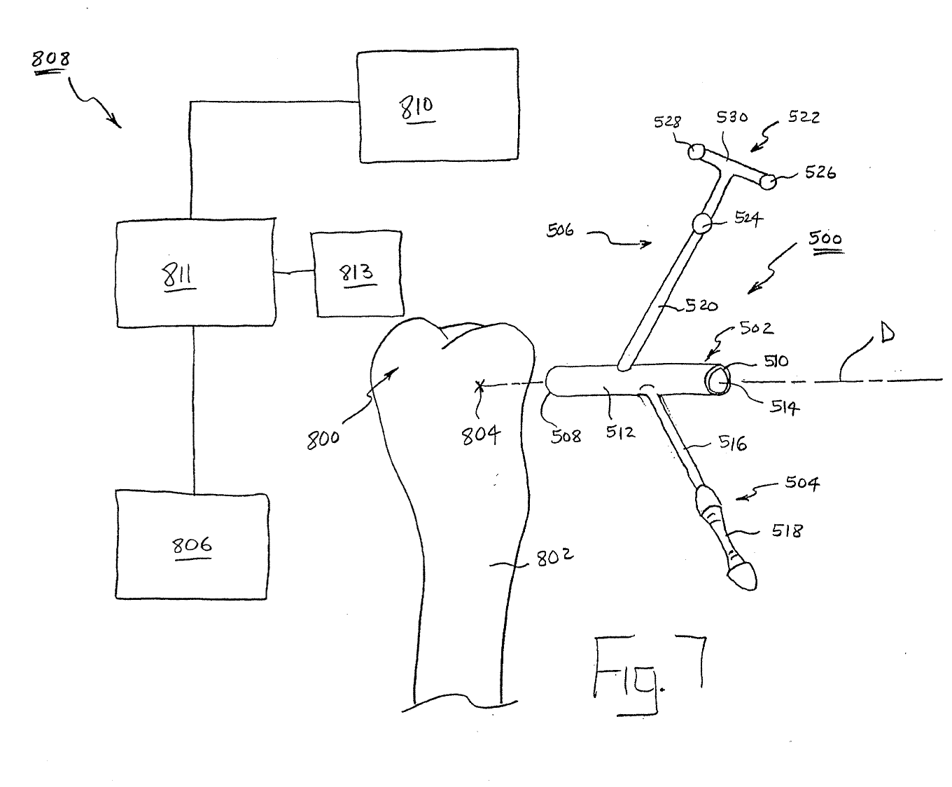

Next, depending upon the embodiment of cutting block 100 used, bores may be created in plane E to receive pins extending from cutting block 100 as depicted in FIG. 9. More specifically, drill cylinder 500 is image guided into the desired orientation relative to plane E to create a first bore at a predetermined target location 820 on plane E in the manner described above. Alternatively, the above-described embodiment having image-guidance assembly 506 connected directly to a drill may be used to create the first bore. Location 820 of the first bore corresponds to the desired location of one of mounting locations 127, 129, such as a pin extending from, or provided in place of, bore 131 of attachment wall 132 when cutting block 100 is in its desired position. After the first bore is created, a second bore is created by image guiding drill cylinder 500 (or the drill itself) into a second position as described above. It should be understood, however, that if the selected cutting block embodiment includes bores instead of pins at mounting locations 127, 129, such as cutting block 100 described above, then drill cylinder 500 may be used to insert two pins into plane E, onto which cutting block 100 is placed. Alternatively, after drill cylinder 500 is used to place a first pin into plane E, a first bore, such as bore 131 of cutting block 100 may be placed onto the first pin and rotated (under image guidance) such that second bore 133 of cutting block 100 registers with a second target location (not shown) on plane E. Then, a second pin may be inserted into plane E through cutting block 100. All of these various procedures for mounting cutting block 100 to plane E are performed in a manner similar to that described above with reference to the procedures for mounting cutting block 100 to distal end 802 of femur 800.

After cutting block 100 is securely in position on the surface of plane E as shown in FIG. 10, the surgeon may make adjustments to the positions of cutting paths 190, 218, 252, 286 of guides 104-110, respectively, such that they correspond to the desired locations of the four bony cuts needed to facilitate placement of the femoral component of the knee implant. Alternatively, the surgeon may employ the above-described iterative process of making a cut, verifying its location and orientation using tracking instrument 600 or 700, and making a new cut if necessary. Assuming image guided adjustment is employed, the surgeon may place plate 720 of tracking instrument 700 into path 190 of guide 104 to determine its precise location relative to the predetermined desired plane (plane F) of the anterior femoral cut. The surgeon may then adjust the position of path 190 until it corresponds precisely to the desired plane F (as indicated on display 806) by rotating grips 400 of adjustors 112, 114, thereby moving guide 104 toward or away from mounting locations 127, 129. Similarly, tracking instrument 700 may be placed into path 218 of guide 110 to permit image guided adjustment (using grips 400 of adjustors 116, 118 in the manner described above) of the position of path 218 relative to the desired plane of the posterior femoral cut (plane G).

The positions of paths 252, 286 of guides 106, 108, respectively, are adjusted in a similar manner. More specifically, tracking instrument 700 may be placed in path 252 of guide 106 to permit image guided adjustment of the lateral position of path 252 relative to mounting locations 127, 129 using grips 402 of linear adjustors 112, 114 in the manner described above. The angular orientation of path 252 may be adjusted (with tracking instrument 700 still in place) by rotating grip 416 of angular adjustor 120 in the manner described above. When display 806 of image guidance system 808 indicates that the actual orientation of path 252 corresponds to the predetermined desired orientation of the inferior anterior chamfer cut (plane H), guide 106 may be locked in place. The same procedure may be used to perform image guided linear and angular adjustment of path 286 of guide 108 to precisely locate the superior posterior chamfer cut (plane I).

After paths 190, 218, 252, 286 are adjusted as described above, the surgeon may use a conventional surgical saw (not shown) to create the four bony cuts (in any order) by moving the saw along paths 190, 218, 252, 286. After the cuts are made, cutting block 100 is removed and the femoral component of the knee implant is positioned and secured to distal end 802 of femur 800 in a conventional manner.

As should be apparent from the foregoing, the present system permits accurate placement of cutting block 100 (on distal end 802 of femur 800 and on plane E) through image guidance of drill cylinder 500. Additionally, the system permits the surgeon to even more precisely locate the bony cuts once cutting block 100 is placed by independently adjusting the positions of one or more of paths 190, 218, 252, 286 relative to mounting locations 127, 129, and, in certain circumstances, the angular orientation of path 252, 286 relative to mounting locations 127, 129 in the manner described above. It should be further understood, however, that pre-operative adjustments of paths 190, 218, 252, 286 may be made to simplify the arthroplasty procedure and reduce the time required for the operation.

More specifically, before the operation, the surgeon or a medical technician may determine the size and style of the prosthetic components based upon the physical characteristics of the patient and the surgeon's preferences. The geometric characteristics of the selected components may then be determined by “tracing” the surfaces of the components with, for example, tracking instrument 600. In other words, while tip 620 is moved along the various surfaces of the components, receiver 810 of image guidance system 808 detects the locations of array 604 and processor 811 of system 808 calculates the corresponding locations of tip 620. Processor 811 processes the data representing the surfaces of the components using any of a variety of conventional techniques to generate a three-dimensional model of the component, which is stored in the memory 813 of system 808. Over time, the models generated in this manner may collectively form a library of models. Of course, manufacturers of prosthetic components may provide on a transportable medium (such as a compact disk) data which describes the various styles and sizes of components they sell. This data may be transferred to system 808 and stored in a library of component models, thereby permitting the surgeon or technician to simply select a component from a menu. The stored three-dimensional model of the selected component is then retrieved to facilitate preoperative adjustment of cutting paths 190, 218, 252, 286 of cutting block 100 as described below.

After, for example, the model of the desired femoral component is selected, the surgeon or technician may “trace” the current configuration of cutting block 100 (i.e., the current positions of paths 190, 218, 252, 286 relative to mounting locations 127, 129 of attachment wall 132. Such “tracing” may be performed by placing plate 720 of tracking instrument 700 (or tip 620 of tracking instrument 600) in paths 190, 218, 252, 286 thereby permitting image guidance system 808 to generate a three-dimensional model of cutting block 100. This data, provided to system 808, permits system 808 to generate an image on display 806 comparing the current positions of paths 190, 218, 252, 286 as they relate to the optimal positions for accommodating the known geometry of the selected femoral component. The surgeon or technician may then sequentially place plate 720 of tracking instrument 700 (or tip 620 of tracking instrument 600) into each of paths 190, 218, 252, 286 and adjust the positions of paths 190, 218, 252, 286 using adjustors 112, 114, 116, 118, 120, 122 in the manner described above. Using this technique, the surgeon may use a first cutting block 100 to create the distal femoral cut (plane E). A second cutting block 100 may then be positioned on plane E using drill cylinder 500 as described above, and the locations of paths 190, 218, 252, 286 (which have been pre-adjusted to correspond to the surfaces of the selected femoral component) may simply be verified by placing plate 720 of tracking instrument 700 (or tip 620 of tracking instrument 600) into each of paths 190, 218, 252, 286 and viewing on display 806 the position of each path 190, 218, 252, 286 relative to the desired position. Of course, if less than optimal placement of cutting block 100 is obtained using drill cylinder 500, then adjustments to the positions and/or angular orientations of paths 190, 218, 252, 286 may be desirable during the operation.

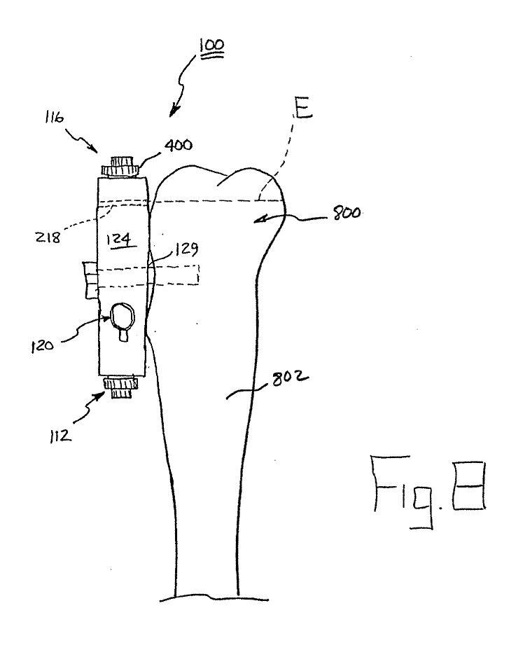

The procedure for preparing the proximal end 900 of the tibia 902 to receive a proximal tibial prosthesis (a tibia tray) is generally described with reference to FIGS. 11-13, and is substantially identical to the procedure described above for making the distal femoral cut (plane E). The above-described alternative embodiments including the image-guided drill (as opposed to drill cylinder 500) and the iterative adjustment process (as opposed to image-guided adjustment) have equal application to the following description of shaping tibia 902.

According to one embodiment, image-guided drill cylinder 500 is first moved by the surgeon into a desired or target location 904 on the anterior surface of proximal end 900 of tibia 902 for placing a pin at one of mounting locations 127, 129 (either directly or percutaneously as described above), which in turn locates cutting block 100. More specifically, while viewing a visual indication on display 806 of the three-dimensional orientation of drill cylinder 500 relative to the known structure of tibia 902 (predetermined by “tracing” the surfaces of tibia 902 using tracking instrument 600 in the manner described above), the surgeon moves drill cylinder 500 (either directly or percutaneously) into contact with target location 904 on proximal end 900 of tibia 902. Target location 904 corresponds to the desired location of one of mounting locations 127, 129 (in this example, one of bores 131, 133 formed in attachment wall 132 of cutting block 100). As explained above, image guidance system 808 provides a visual indication on display 806 based on data representing the three-dimensional structure of tibia 902, drill cylinder 500, and cutting block 100. Receiver 810 of image guidance system 808 detects the position of elements 524, 526, 528 of array 522, thereby enabling system 808 to accurately determine the location of end 508 of drill cylinder 500 relative to target location 904. In this manner, the surgeon is able to determine not only whether the point of entry of the pin (not shown) is accurately located, but also whether the angle of entry (represented by axis D in FIG. 11) is appropriate.

Once drill cylinder 500 is accurately placed, an appropriately sized pin is placed into central bore 514 of drill cylinder 500 and screwed or otherwise inserted into target location 904 of tibia 902. The depth of entry of the pin may be controlled using any of a variety of conventional techniques. After the pin is placed, drill cylinder 500 is removed. It should be understood, however, that instead of placing a pin into tibia 902 as described above, drill cylinder 500 may be used to guide a drill bit (not shown) for creating a bore into tibia 902. The depth of entry of the drill bit may similarly be controlled using any of a variety of conventional techniques. After the bore is created and drill cylinder 500 is removed, an appropriately sized pin may be securely inserted into the bore. One of bores 131, 133 of corresponding cutting block 100 in such an embodiment would be sized to securely receive the pin. As yet another alternative, drill cylinder 500 may be used in the manner described above to create a bore in tibia 902 for receiving a pin connected to cutting block 100 at one of mounting locations 127, 129.

Assuming a first pin is placed into the tibia according to the procedure described above, a second pin (not shown) may be placed by repeating the procedure, but placing the second pin at a second target location (not shown) that corresponds to the other of mounting locations 127, 129 (in this example, bores 131, 133) in the known geometry of cutting block 100. After the second pin is inserted into the second target location, drill cylinder 500 is removed. Next, bores 131, 133 of cutting block 100 are positioned onto the first and second pins. As described above, any of a plurality of different techniques may be used to secure cutting block 100 to the pins.

In an alternative embodiment, after the first pin is placed according to the procedure described above, one of bores 131, 133 of cutting block 100 may be placed on the first pin and rotated into position under image guidance such that the other of bores 131, 133 of cutting block 100 registers with the second target location to permit creation of a second bore in proximal end 900 of tibia 902 or insertion of a second pin in the manner described above.

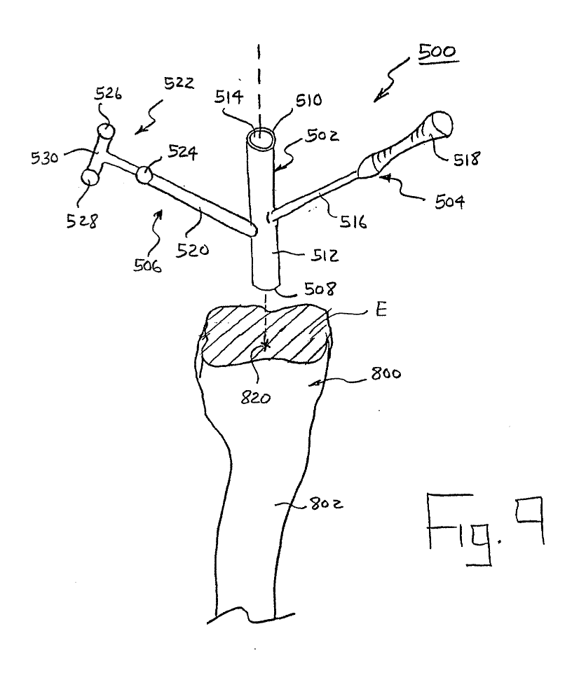

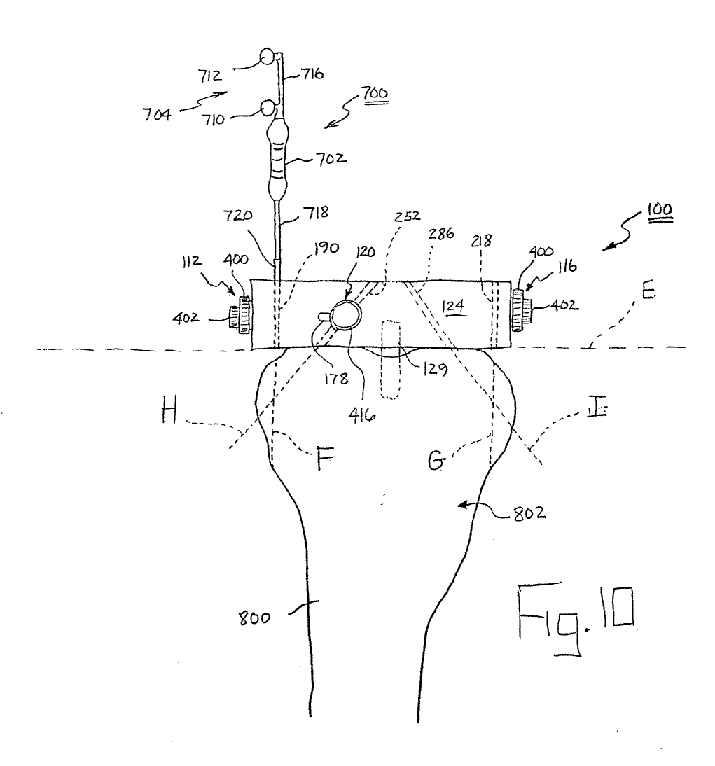

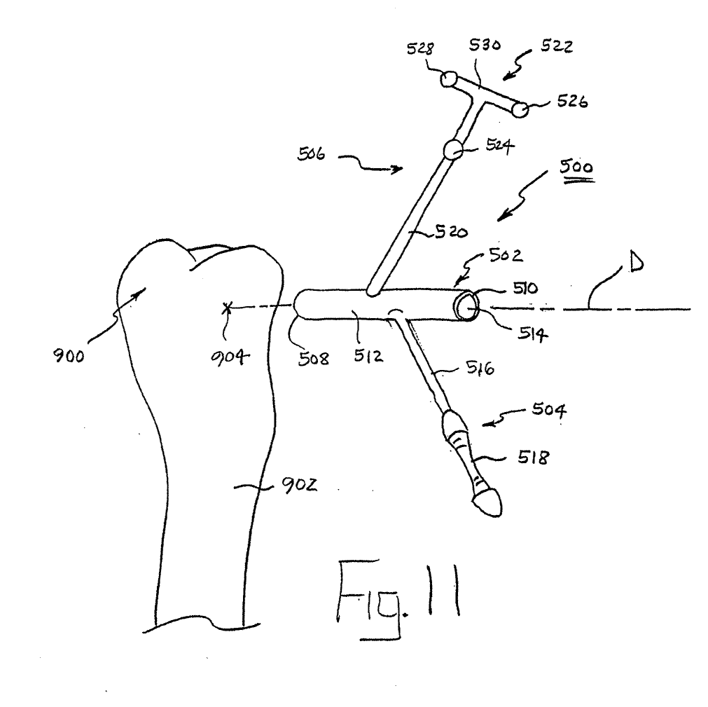

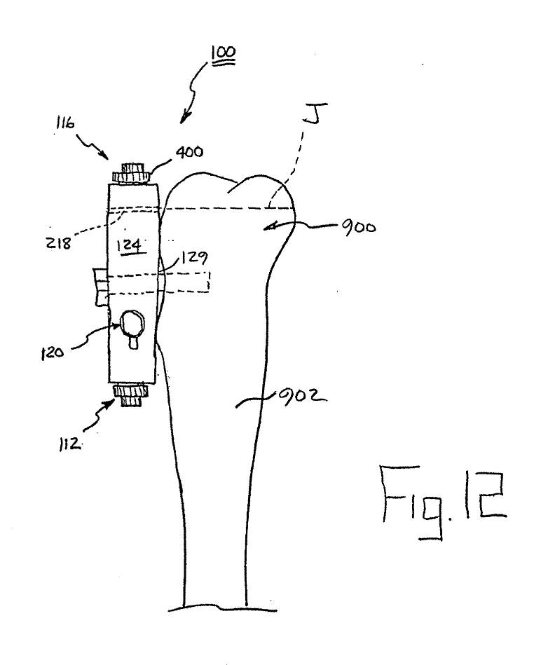

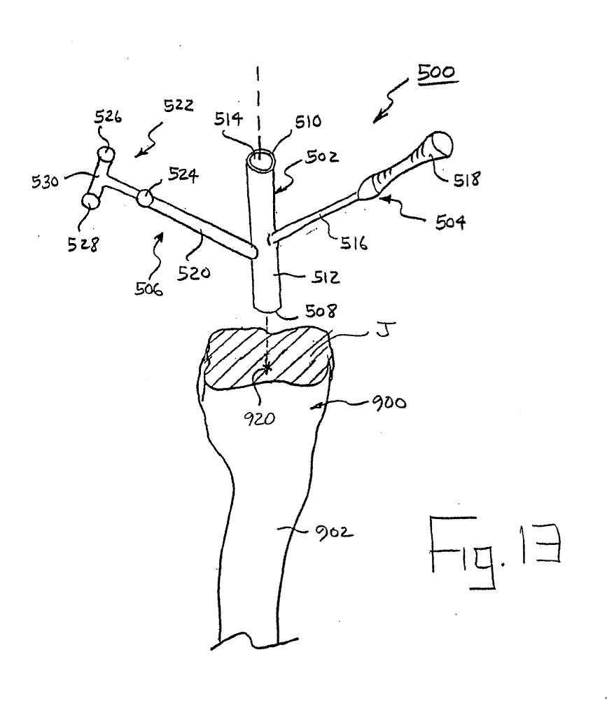

After cutting block 100 is secured on the pins (as shown in FIG. 12), the position of a cutting path, such as path 218, may be adjusted to correspond precisely to the desired orientation of the proximal tibial cut (plane J). Like the femoral cuts described above, the precise orientation of plane J varies based on the geometry of the tibial tray selected, the physical characteristics of the patient, and the preferences of the surgeon. Typically, the medial/lateral orientation of plane J is substantially perpendicular to the tibial mechanical axis, and the anterior/posterior orientation ranges from zero to twelve degrees of posterior slope. The geometry of the selected tibial component is “traced” or otherwise entered into system 808 either during the procedure or pre-operatively as described above. The actual position of path 218 may be determined by placing plate 720 of tracking instrument 700 (or tip 620 of tracking instrument 600) into path 218. System 808 may then process the known geometry of the tibial component, the known geometry of cutting block 100, and the current position of path 218 to provide an image on display 806 to indicate whether path 218 corresponds with sufficient accuracy to the desired location and orientation of plane J. If not, the surgeon may adjust the position of path 218 in the manner described above. Finally, after plane J is created, the surgeon removes cutting block 100.

Referring now to FIG. 13, the surgeon may next use drill cylinder 500 in the manner described above to accurately locate a bore into plane J at a target location 920 corresponding to a post that typically extends from the tibial component for attachment of the component to tibia 902. Once drill cylinder 500 is accurately located under image guidance, a drill bit (not shown) is passed through central bore 514 to create the bore at target location 920. Finally, drill cylinder 500 is removed, and the surgeon installs and secures the tibial component using any of a variety of conventional techniques. The remaining steps for completion of the arthroplasty procedure do not involve use of the present system.

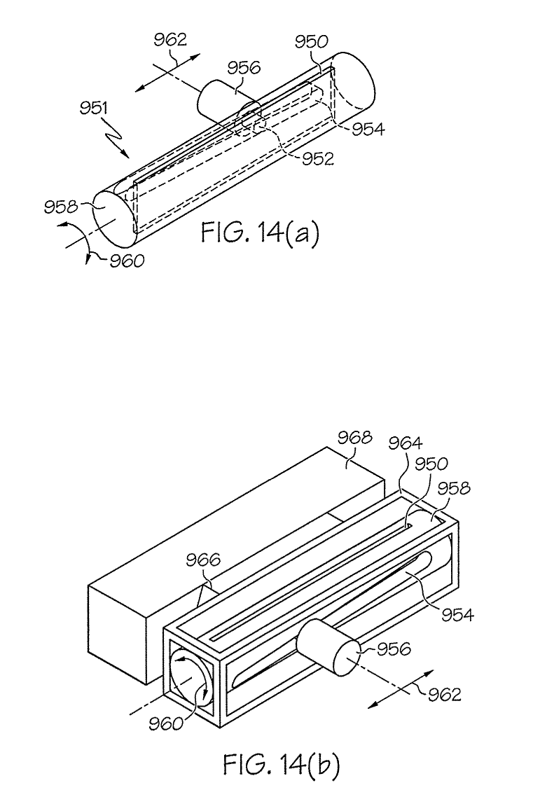

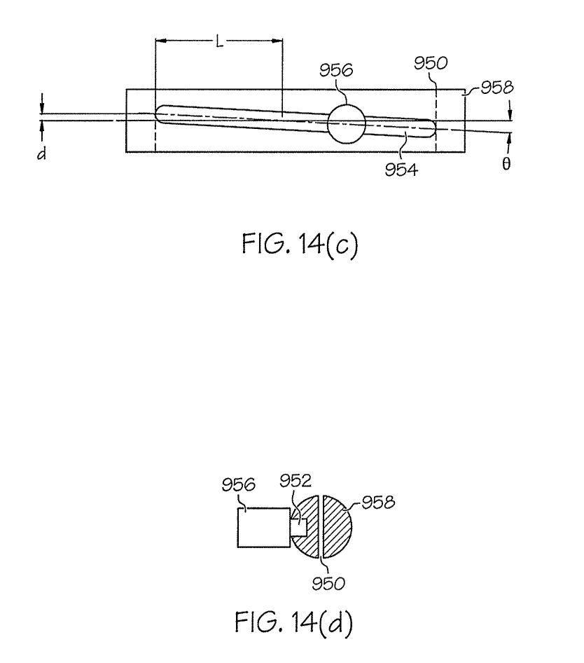

FIGS. 14a-d depict one illustrative embodiment of a fine adjustment bone cutting apparatus that can be used in conjunction with the bone cutting methods of the present teachings. Generally speaking, the bone cutting apparatus has a fine adjustment block 951 that allows for fine angle adjustment (relative to a resection plane) of conventional instruments or jigs, as well as stand-alone assemblies. The fine adjustment block 951 is formed of a cylindrical housing 958, having a resection slot 950 that is placed perpendicular to its long axis. The housing is configured to rotate about a rotational axis 960 in such a manner that the miter or cutting angle of the resection slot 950 can be changed or fine adjusted relative to a resection plane. This movement is accomplished by sliding or translating a cam knob or dial 956 from side-to-side along a diagonal cam slot 954. To accommodate this movement, the cam knob 956 has a small tip or cam pin 952 at one end that is slidably held within the cam slot 954. As the cam knob 956 is moved side-to-side along a translational axis 962, the cam pin 952 forces the cylindrical housing 958 to rotate about the rotational axis 960.

Due to the mechanical nature of the cylindrical housing 958, the resection slot 950 will not rotate about the rotational axis 960 unless the cam knob 956 is moved or translated from side-to-side. More particularly, the cam knob 956 is configured to be locked relative to the cylindrical housing 958 to ensure rigidity of the resection slot 950 during a resection procedure. For instance, in one exemplary embodiment, the knob's cam pin 952 is threaded such that when the knob 956 is turned, the threaded portion advances into the cam slot 954 and ultimately presses against its inner wall. Once the threaded portion encounters sufficient resistance from the inner wall of the cam slot 954, the knob 956 is prevented from translational movement within the cam slot.

As shown in FIG. 14b, the cylindrical housing 958 may also be enclosed within a frame structure 964 that is removably attached or connected to a stand-alone assembly 968 (such as a cutting guide) by one or more connectors 966 to assist with the adjustability of the apparatus during a resection procedure.

The side-to-side movement of the cam knob 956 is limited to movement along the translational axis 962 within the diagonal cam slot 954, as shown in FIGS. 14b and 14c. More particularly, the cam knob 956 is configured such that it can only move along the direction of the arrow representing translational axis 962. Thus, as the cam knob 956 is moved from side-to-side, the cam pin 952 engages the cam slot 954 and forces the cylindrical housing 958 to rotate back and forth, thereby changing the angle of the resection slot 950 relative to the rest of the cutting apparatus. This movement can be quantified mathematically, wherein θ represents the rotational angle that the resection slot 950 will change (+/−) as the cam knob 956 is translated within the cam slot 954 from side-to-side, L represents ½ the length of the cam slot 954 and d represents the slot height such that:

$${tan}^{- 1} θ = \frac{d}{L} .$$

The rotational adjustability of the cutting apparatus is further shown in FIG. 14d, where a cross-section of the cylindrical housing 958 is illustrated. The cam knob 956 connects to the cylindrical housing 958 by way of the cam pin 952, which is held within the housing. As explained above, as the cam knob 956 is moved from side-to-side within the diagonal cam slot 954, the cam pin 952 engages the inner portion of the cam slot 954 and forces the cylindrical housing 958 to rotate about the rotational axis 960 such that the angle of the resection slot 950 is altered with respect to the resection plane. This rotational movement allows the surgeon to fine adjust the angular position of the cutting block relative to the resection plane during a resection procedure.

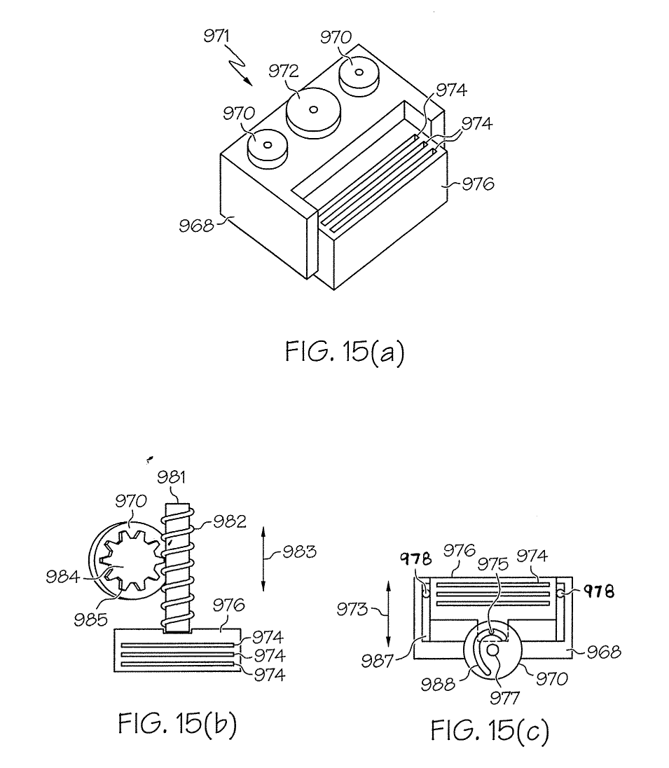

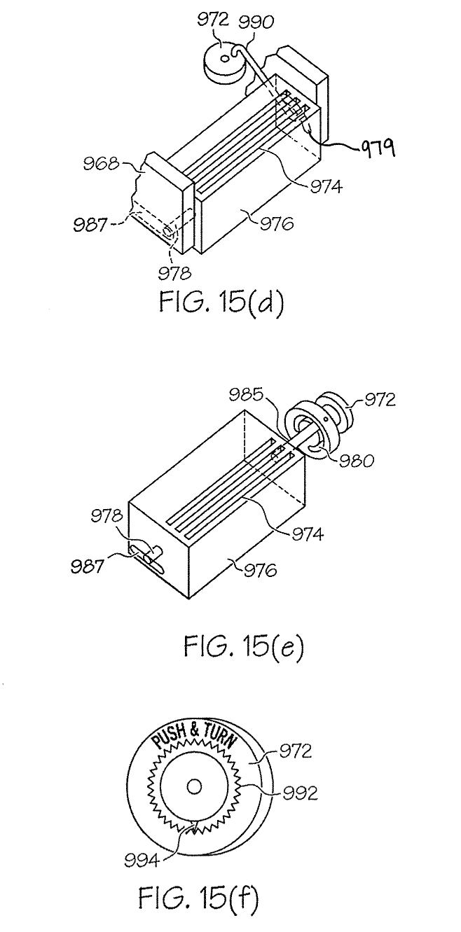

Another embodiment of a fine adjustment bone cutting apparatus is shown in FIGS. 15a-f. This exemplary apparatus provides fine adjustment adaptability to conventional 4-in-1 cutting blocks. More particularly, this illustrative embodiment allows a surgeon to attach a cutting block to a patient and then fine tune its location to best match the patient's femoral rotation landmark, whether such landmark is chosen by the surgeon or a surgical navigation system. To accomplish this, the assembly comprises micro adjustment knobs that are capable of fine adjusting the position of the cutting block on the patient's anatomy. More particularly, in the illustrated embodiment of FIG. 15a, an adjustment assembly 971 includes an assembly housing 968 that is connected to a resection block 976 (such as a 4-in-1 resection block) and has one or more adjustment knobs (e.g., 970, 972) that are configured to change the direction and/or angular position of the cutting block and its resection slots 974 after the block is placed on the patient's anatomy. For instance, the assembly housing 968 may include axial dials 970 and/or angular rotation dials 972. These dials can be independently turned so that the axial position (e.g., the medial/lateral and/or anterior/posterior position of the block) and/or the rotational alignment of the resection block is fine adjusted with respect to the patient's anatomy.

Several embodiments for moving the resection block 976 are shown in FIGS. 15b-f. For instance, FIG. 15b shows a bottom view of an axial movement embodiment in which the cutting block is moved in a linear direction (shown by arrow 983) after it is placed on the patient's anatomy. According to this illustrative embodiment, one of the axial dials 970 is fixably connected to an axial wheel 984 having a plurality of teeth 985 along its outer periphery. The teeth 985 of the axial wheel 984 are further meshed or aligned with threads 982 on a worm gear 981. In turn, the worm gear 981 is fixably connected to the resection block 976. When the axial dial 970 is rotated, the axial wheel 984 rotates with it. As the axial wheel 984 rotates, its teeth 985 forcibly engage the worm gear's threads 982 and cause the worm gear 981 to move the resection block 976 in a linear direction representative of arrow 983, the direction depending on which way the dial 970 is turned. This axial movement allows the surgeon to fine tune the position of the cutting block 976 and its resection slots 974 relative to a patient's anatomy to more accurately conduct a distal resection procedure, for instance.

Another axial movement embodiment is shown in FIG. 15c. According to this embodiment, the axial dial 970 is connected to the resection block 976 by way of a fixed cam arm 975. To achieve axial movement of the resection block 976, the fixed cam arm 975 has a small tip or cam pin at its upper end that is slidably held within a cam slot 988 associated with the axial dial 970. As the axial dial 970 is rotated about a rotatory axis, the cam pin engages the inner surface of the cam slot 988 and causes the cam arm 975 to translate an axial force on the resection block 976 as the cam pin travels along the curvature of the cam slot 988. More particularly, the curvature of the cam slot 988 is shaped in such a manner that as the cam pin travels along the curvature of the cam slot as the axial dial 970 is rotated, the distance that the tip of the cam arm 975 is located from a center post 977 on the axial dial is either increased or decreased (depending on the direction the dial is rotated). The rotary motion of the cam pin within the cam slot 988 is then translated into linear motion by placing a force on the resection block so that it is moved away from the assembly housing 968 along the direction of arrow 973. The linear distance that the resection block 976 is capable of moving with respect to the assembly housing directly corresponds to the distance that the cam arm 975 changes with respect to the center post 977 (i.e., increase or decrease) during the rotation of the axial dial 970. To achieve the linear movement of the resection block 976 as described above, two axial pins 978 are slidably held within axial tracks 987 coupled to the resection block. As the cam arm 975 engages the resection block after being activated by the rotated axial dial 970, the axial pins 978 are caused to travel along the axial tracks 987 and thereby move the resection block linearly with respect to the assembly housing.

A rotational movement embodiment in accordance with the present teachings is shown in FIG. 15d. In this illustrative embodiment, the rotational dial 972 of the adjustment assembly is connected to a rotational linkage arm 990 that couples to the resection block 976 inside a rotational cam slot 979. As the rotational dial 972 is turned, the rotational linkage arm 990 is also caused to move because of its fixed relationship with the rotational dial 972. That is, the end of arm 990 rotates relative to a cylindrical recess of the rotational dial 972 (not shown) in which it is inserted while the long portion of the arm 990 travels along the inner surface of the rotational cam slot 979. To achieve rotational movement of the resection block 976, the rotational linkage arm 990 also has a small tip or cam pin that is slidably held within the rotational cam slot 979 of the resection block. As the rotational dial 972 is rotated about a rotatory axis, the cam pin engages the inner surface of the rotational cam slot 979 and causes the linkage arm 990 to translate a rotational force on the resection block 976 and cause it to tip forward as the cam pin travels along the inner curvature of the cam slot 979.

In addition to moving the resection block 976 rotationally, in certain embodiments, the adjustment assembly 971 may also comprise an axial/linear movement assembly, such as discussed above with reference to FIGS. 15b and 15c. Depending on the resection procedure that is being performed, the chronological order in which the block is adjusted (i.e., axially or rotationally) can be arranged as necessary. For instance, in certain embodiments, the axial movement of the resection block is set first and then the rotational angle of the block configured. After the appropriate axial position of the block is achieved with respect to a desired resection plane, the block can then be locked into place by turning a locking knob or ratchet mechanism, such as shown and discussed below with respect to FIG. 15(f). After the position of resection block 976 is locked axially, the block can then be adjusted rotationally as discussed above. In other embodiments, the rotational angle of the block is set before the linear/axial position of the block is determined. According to this embodiment, the axial knob can be slidably held within a linear slot (not shown) so that once the rotational angle of the block is locked into place, the block will be able to be adjusted linearly/axially without disrupting or changing the angular position of the block. More particularly, the slot will permit the knob to travel together with the block as it is translated linearly in the direction of arrow 973.

Another rotational movement embodiment in accordance with the present teachings is depicted in FIG. 15e. According to this illustrative embodiment, the rotational dial 972 is connected directly to an internal dial having a rotational cam slot 980, which in turn is connected to a fixed rotational pin 985 that is attached to the resection block 976. By turning the rotational dial 972, the internal dial is also caused to rotate. As the internal dial is rotated, the rotational pin 985 advances along its inner surface, resulting in the resection block 976 rotating or tipping forward because of their fixed relationship. More particularly, as the rotational dial 972 is rotated about a rotatory axis, a cam pin (not shown) at the end of the rotational pin 985 engages the inner surface of the rotational cam slot 980 and causes the rotational pin 985 to translate a rotational force on the resection block 976 and cause it to tip forward as the cam pin travels along the inner curvature of the cam slot 980. This rotational movement allows the surgeon to fine tune the angular position of the cutting block 976 and its resection slots 974 relative to a patient's anatomy to more accurately plan the depth of a distal resection, for instance.

In addition to moving the resection block 976 rotationally, in certain embodiments, the adjustment assembly may also comprise an axial/linear movement assembly, such as discussed above with reference to FIGS. 15b and 15c. Depending on the resection procedure that is being performed, the chronological order in which the block is adjusted (i.e., axially or rotationally) can be planned accordingly. To achieve the linear movement of the resection block 976, two axial pins 978 are slidably held within axial tracks 987 coupled to the resection block.

FIG. 15f depicts a ratchet mechanism that can be used in conjunction with any of the above described fine adjustment apparatus embodiments to lock the resection block in a fixed position axially and/or rotationally during a fine adjustment procedure. In this illustrative embodiment, when the rotational dial 972 is depressed, the internal gears of the rotational dial 972 are caused to mesh with a ratchet gear 992 and cause a ratchet stop 994 to push down (and out of the way) the ratchet gear 992. In the up (normal) position, the ratchet stop 994 is configured to hold the ratchet gear 992 into place. Moreover, a combination of coil, leaf, and torsion springs may also be used to position the axial dials 970 and the rotational dial 972 in a resting position. Similarly, the ratchet gear 992 can be forced back to a resting position by using a torsion spring.

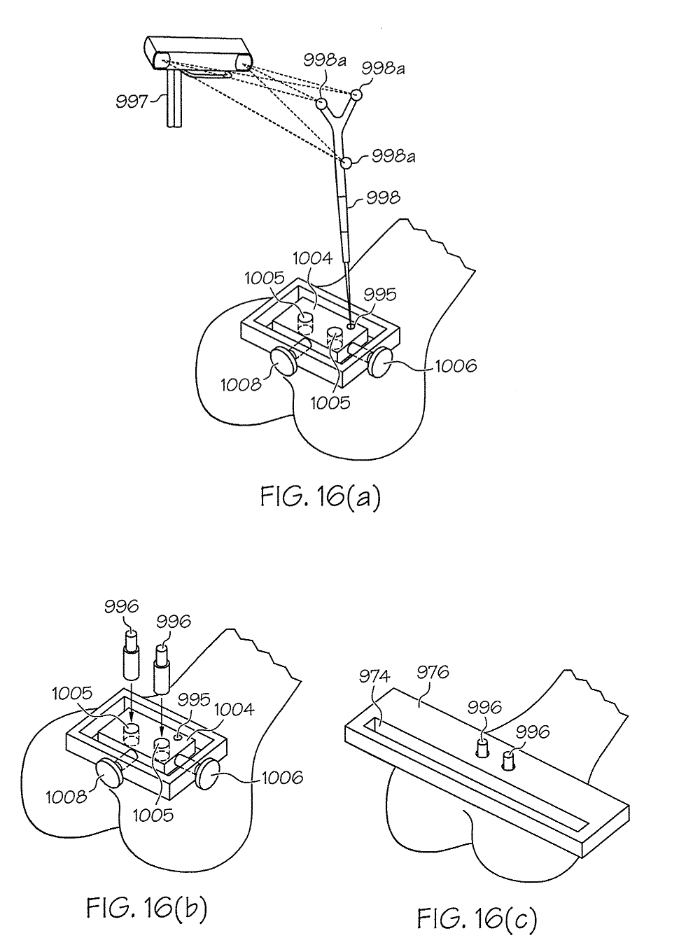

Another fine adjustment apparatus in accordance with the present teachings is shown in FIGS. 16a-16c. This adjustable apparatus is configured to allow the correct placement of pins into a patient's bones such that any resection instrument can be appropriately aligned (e.g., correct angle and location) with the patient's anatomy. Turning in greater detail to FIG. 16a, a resection block pin assembly 1004 is shown having a longitudinal adjustment dial 1006 and axial adjustment dial 1008, both of which are configured to adjust the relative position (e.g., varus/valgus and flexion angle) of the resection block pin assembly with respect to the patient's anatomy. To achieve the fine adjustment of the pin assembly 1004, the surgeon attaches the assembly to the patient's anatomy and then fine tunes the position of the assembly by turning the respective dials 1006, 1008 as needed. Once the assembly is appropriately fine tuned to match the stabilizing holes of the resection instrument that is to be used during the resection procedure, one or more pins 996 are inserted into the pin holes 1005 of the assembly to affix it to the patient's anatomy (see FIG. 16b). After the pins 996 are inserted into the assembly 1004, the assembly can then be removed and the resection instrument or device slid over the stabilizing pins. At this point, no further fine-adjustment is needed.

The pin assembly 1004 can also be used in conjunction with a surgical navigation procedure, in which a probe array 998 is introduced to determine the necessary adjustments (relative to a cut plane or the drill hole itself) which must be made to the pin assembly to match a particular anatomy and/or the resection instrument. To track the relative position of the assembly as it is fine adjusted, the tip of the probe array 998 is inserted into a hole or slot 995 on the surface of the assembly and a tracking system locates and tracks the probe array 998 in real-time by using an optical camera 997 as the assembly 1004 is fine adjusted. More particularly, the tracking system determines the position of the probe array by detecting the position of markers 998a on the probe array 998 in space using triangulation methods known within the art. The relative location of the probe array 998 can then be shown as a surgical plan image on a computer display within the surgical field. Once the pins 996 have been correctly positioned, the pin assembly is removed and the resection instrument 976 is introduced over the pins as needed. Thereafter, a bone cutting device can be inserted into the resection instrument's resection slot 974 and the patient's bone resected.

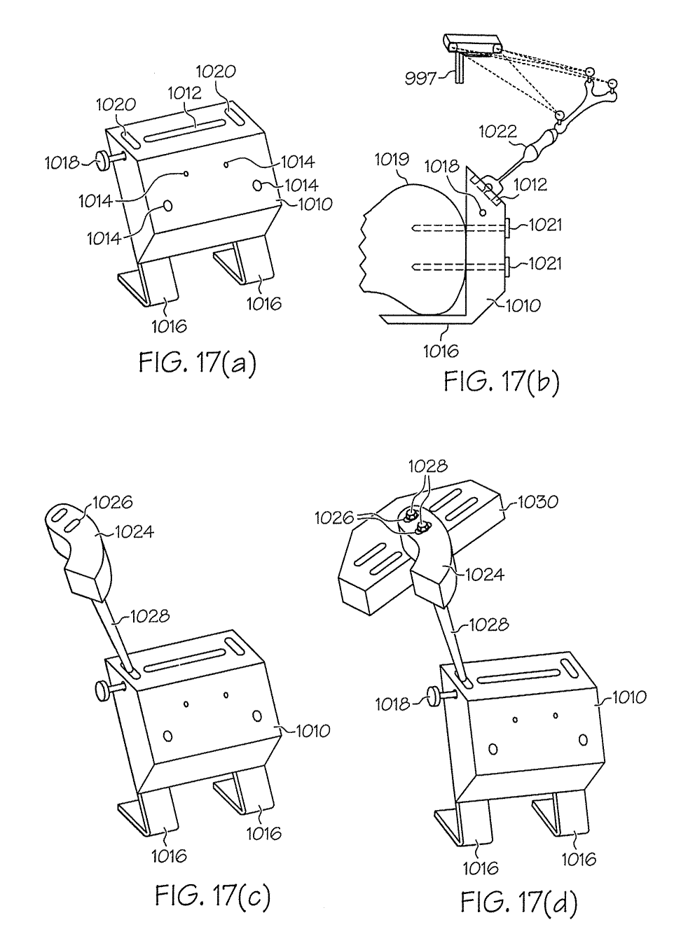

Yet another fine adjustment apparatus in accordance with the present teachings is shown in FIGS. 17a-d. According to this illustrated embodiment, a surgeon fits a cutting block 1010 over a bone 1019 (with the assistance of brackets 1016) and then determines its position using surgical navigation techniques and modifies its position if necessary. By utilizing intramedullary (“IM”) femur instrumentation, the surgeon can perform standard techniques and then check the block placement with a spatula probe 1022 while having the cutting block securely attached to the IM rod. The cutting block 1010 can then be adjusted in varus/valgus, resection height and flexion/extension. The surgeon can adjust for one degree of freedom at a time by placing the spatula probe 1022 in a spatula probe slot 1012 and then monitoring the position of the spatula probe 1022 via a camera 997 that is associated with the surgical navigation system. Once the desirable position has been achieved, the surgeon attaches the block 1010 to the bone 1019 with attachment screws 1021 through attachment holes 1014.

In addition to IM procedures, the present fine adjustment apparatus of FIGS. 17a-d can also be used with extramedullary (“EM”) procedures. According to this embodiment, once the cutting block 1010 is firmly attached to the bone 1019, the surgeon places an outrigger device 1024 onto the cutting block and the flexion/extension orientation is modified by a pivotable extension rod 1028 that is held within an outrigger extension rod slot 1020. The outrigger 1024 is configured to rotate through its connection with the cutting block 1010 and is tracked by the navigation system by placing the spatula probe 1022 in the distal cutting slot. The rotation of the device is then set and is secured into place by tightening the outrigger extension rod screw 1018.

The surgeon can also attach a 4-in-1 cutting block 1030 (or other such cutting block device) to the outrigger 1024 via one or more outrigger fasteners 1028 through outrigger slots 1026. After the 4-in-1 cutting block 1030 is attached, the surgeon may then use the blade slots available on the block to saw appropriate sections of the bone as needed.

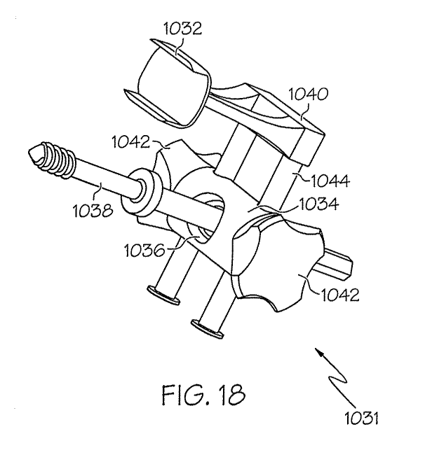

Still yet another embodiment of the present teachings is depicted in FIG. 18. According to this embodiment, a surgeon uses a cut block manipulator 1031 to aid in placing a bone cutting block. The bone cutting block is attachable to the cut block manipulator 1031 via a block attachment face 1032 that includes a series of magnets or other generally known fastening devices. The surgeon vertically adjusts the cut block attachment face 1032 so as to achieve the desired position for placing the attachment instrument. The cut block attachment face 1032 is attachable to a manipulator arm 1040, which in turn attaches to sliding rods 1044 that run through the body 1034 of the manipulator 1031. The body 1034 houses a spherical center section 1036, which allows a lag screw 1038 to rotate 3-Dimensionally. Moreover, the body 1034 is configured to apply a tension to the spherical center section 1036 to thereby hold it into place once it is appropriately positioned by adjustable dials 1042. Once the surgeon is satisfied with the position of the cut block manipulator 1031, the surgeon inserts the lag screw into the bone.

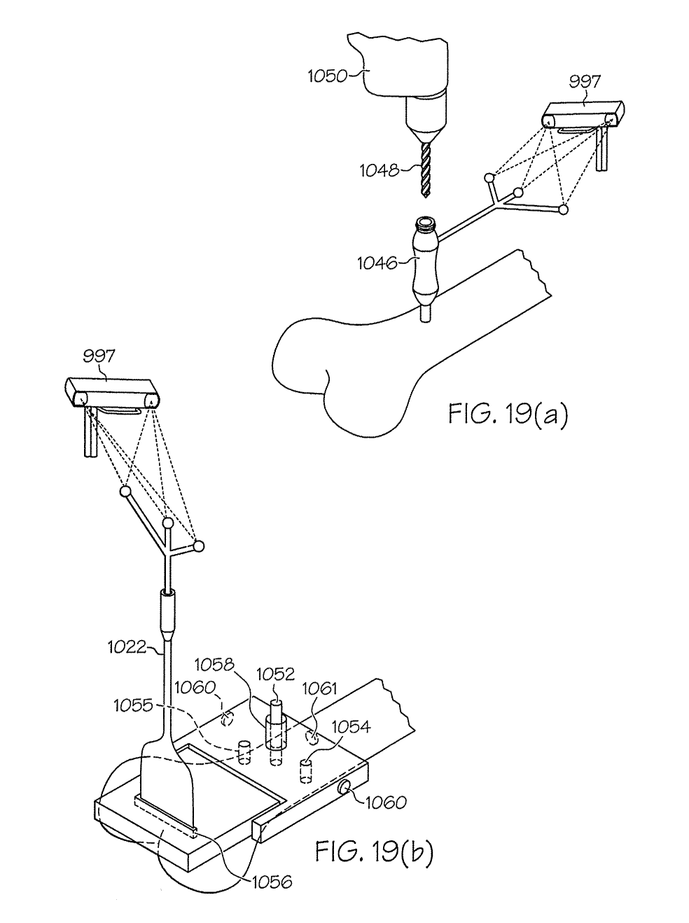

Another embodiment of a fine adjustment apparatus is shown in FIGS. 19a-b. According to this embodiment, a surgeon uses a drill guide 1046, drill 1050, and drill bit 1048 to drill a hole in the patient's bone. The position of drill guide 1046 (and/or alternatively the drill 1050) is tracked by a camera 997 associated with the surgical navigation tracking system. The surgeon then places a pin 1052 into the newly drilled hole and places a cutting block assembly with a cylindrical guide 1058 over the pin 1052 and adjusts one or more dials 1060, 1061 to achieve the appropriate varus/valgus angles. Once the surgeon determines the correct placement of the cutting block assembly by using a spatula array 1022 tracked by the camera 997, the surgeon drills a second hole through the cutting block drill guide 1054 and places a pin into the bone. The surgeon then inserts a third pin by following the same procedure as described above. In certain embodiments, the third pin is placed manually without using surgical tracking methods, while in other embodiments the third pin is placed by using a second drill guide 1055. In certain embodiments, the adjustable dials 1060, 1061 may be configured to make an audible sound when their positions are changed.

The bone cutting apparatus and methods of the present teachings can also be embodied on a computer readable storage medium and operate with the assistance of one or more software programs. According to these embodiments, the computer readable storage medium stores instructions that, when executed by a computer, cause the surgical navigation system to perform a fine adjustment process. The computer readable storage medium can be any medium suitable for storing instruction that can be executed by a computer such as a compact disc (CD), digital video disc (DVD), flash solid-state memory, hard drive disc, floppy disc, and the like.

While exemplary embodiments incorporating the principles of the present teachings have been disclosed hereinabove, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain and which fall within the limits of the appended claims.

What is claimed is:

1. A bone cutting apparatus configured to guide a surgical instrument during a bone cutting procedure, comprising:
a cutting guide having a resection slot, the cutting guide being attachable to a bone; and
a fine adjustment block coupled to the cutting guide, the fine adjustment block having a first adjustment dial configured to change the position of the resection slot relative to a resection plane.

2. The bone cutting apparatus of claim 1, further comprising a first slot configured to receive the first adjustment dial and accommodate linear movement of the resection slot relative to the resection plane.

3. The bone cutting apparatus of claim 2, further comprising a second adjustment dial coupled to the fine adjustment block, the second adjustment dial being configured to change the position of the resection slot relative to the resection plane.

4. The bone cutting apparatus of claim 3, further comprising a second slot configured to receive the second adjustment dial and accommodate angular adjustment of the resection slot relative to the resection plane.

5. The bone cutting apparatus of claim 1, further comprising a tracking element attachable thereto, the tracking element being detectable and trackable by a surgical navigation system.

6. The bone cutting apparatus of claim 1, further comprising a locking mechanism configured to lock the position of the resection slot relative to a resection plane.

7. A method of fine adjusting a bone cutting apparatus during a surgical navigation procedure, comprising:
attaching a cutting guide assembly to a bone, the cutting guide assembly having a resection slot;
adjusting the position of the resection slot relative to a predetermined resection plane by moving at least one adjustment dial on the cutting guide assembly; and
aligning the resection slot with the predetermined resection plane by image guiding a tracking element associated with the cutting guide assembly.

8. The method of claim 7, wherein adjusting the position of the resection slot comprises moving the cutting guide assembly from a first position to a second position linearly relative to the predetermined resection plane.

9. The method of claim 7, wherein adjusting the position of the resection slot comprises rotating the cutting guide assembly from a first position to a second position relative to the predetermined resection plane.

10. The method of claim 9, wherein rotating the cutting guide assembly comprises causing angular movement of the resection slot relative to the predetermined resection plane.

11. A method of fine adjusting an instrument during a surgical navigation procedure, comprising:
positioning a drill guide assembly against a bone, the drill guide assembly having at least one drill hole;
incorporating a surgical tool into the drill guide assembly, the surgical tool having a tracking element detectable by a surgical navigation tracking system;
adjusting the position of the drill guide assembly so that the at least one drill hole substantially corresponds with a target location on the bone;
drilling one or more holes into the bone at the target location;
inserting one or more guide pins into the one or more holes; and
placing a resection instrument over the one or more guide pins.

12. The method of claim 11, wherein incorporating the surgical tool into the drill guide assembly comprises inserting a tip of the surgical tool into a slot on the drill guide assembly.

13. The method of claim 12, wherein the surgical tool comprises a surgical probe.

14. The method of claim 11, wherein adjusting the position of the drill guide assembly comprises moving the drill guide assembly from a first position to a second position linearly relative to the target location.

15. The method of claim 11, wherein adjusting the position of the drill guide assembly comprises rotating the drill guide assembly from a first position to a second position relative to the target location.

16. The method of claim 15, wherein rotating the drill guide assembly comprises causing angular movement of the resection slot relative to the target location.

17. The method of claim 11, wherein adjusting the position of the drill guide assembly comprises turning a dial on the drill guide assembly.

18. The method of claim 17, further comprising using software associated with the surgical navigation tracking system to determine how much the dial is to be turned to position the drill guide assembly relative to the target location.

19. The method of claim 11, wherein the resection instrument comprises a cutting block.

20. The method of claim 11, wherein the target location comprises a predetermined resection plane.
